(19) United States
(12) Patent Application Publication
(10) Pub. No.: US 2019/0076158 A1
(43) Pub. Date: Mar. 14, 2019

(54) NOSE CLEANING ASSEMBLY

(71) Applicant: Sharon Cummings, Mukilteo, WA (US)

(72) Inventor: Sharon Cummings, Mukilteo, WA (US)

(21) Appl. No.: 15/704,450

(22) Filed: Sep. 14, 2017

(51) Int. Cl. A61B 17/24; A61F 13/38

(52) U.S. Cl. CPC A61B 17/24; A61B 2017/246; A61F 13/38

(57) ABSTRACT

A nose cleaning assembly for removing mucous and hardened deposits from a nostril includes at least one rod. The at least one rod is configured to be grasped in digits of a hand of a user to selectively position opposing ends of the at least one rod to into a nostril of the user. At least one swab is coupled to a respective opposing end of the at least one rod. At least one plate is coupled to a respective opposing end of the at least one rod. The at least one swab is configured to absorb mucous that is positioned in the nostril to remove the mucous from the nostril. The at least one plate is configured to scrape hardened deposits that are positioned in the nostril to remove the hardened deposits from the nostril.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to cleaning assemblies and more particularly pertains to a new cleaning assembly for removing mucous and hardened deposits from a nostril.

### BRIEF SUMMARY OF THE INVENTION

An embodiment of the disclosure meets the needs presented above by generally comprising at least one rod. The at least one rod is configured to be grasped in digits of a hand of a user to selectively position opposing ends of the at least one rod to into a nostril of the user. At least one swab is coupled to a respective opposing end of the at least one rod. At least one plate is coupled to a respective opposing end of the at least one rod. The at least one swab is configured to absorb mucous that is positioned in the nostril to remove the mucous from the nostril. The at least one plate is configured to scrape hardened deposits that are positioned in the nostril to remove the hardened deposits from the nostril.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a nose cleaning assembly according to an embodiment of the disclosure.

FIG. 2 is a top view of an embodiment of the disclosure.

FIG. 3 is a front view of an embodiment of the disclosure.

FIG. 4 is a side view of an embodiment of the disclosure.

FIG. 5 is a top view of an embodiment of the disclosure.

FIG. 6 is a front view of an embodiment of the disclosure.

### DETAILED DESCRIPTION OF THE INVENTION

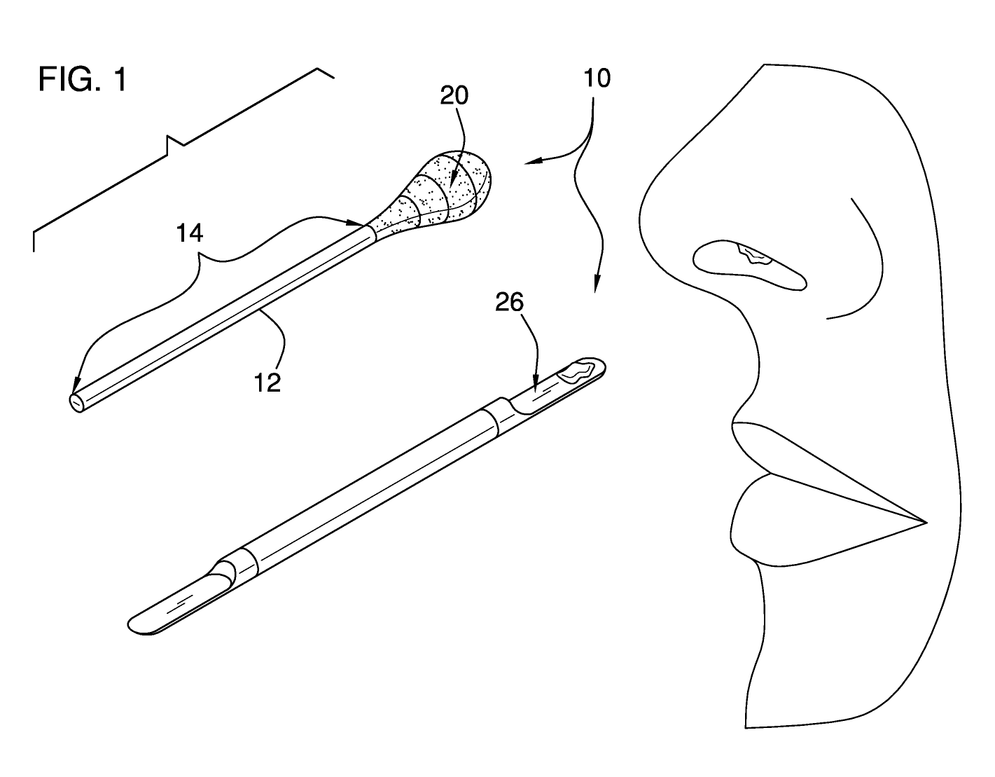

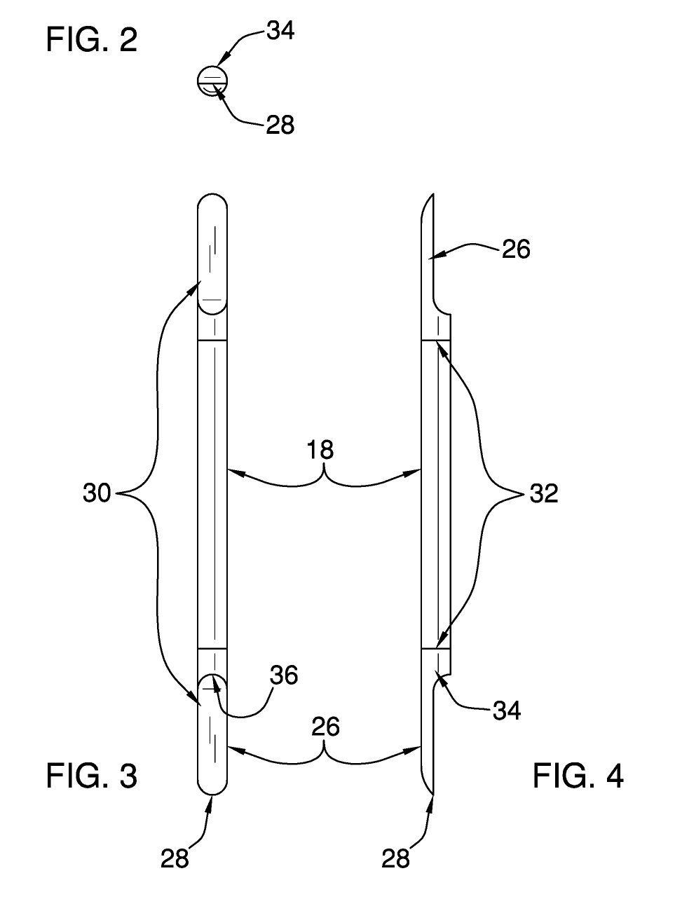

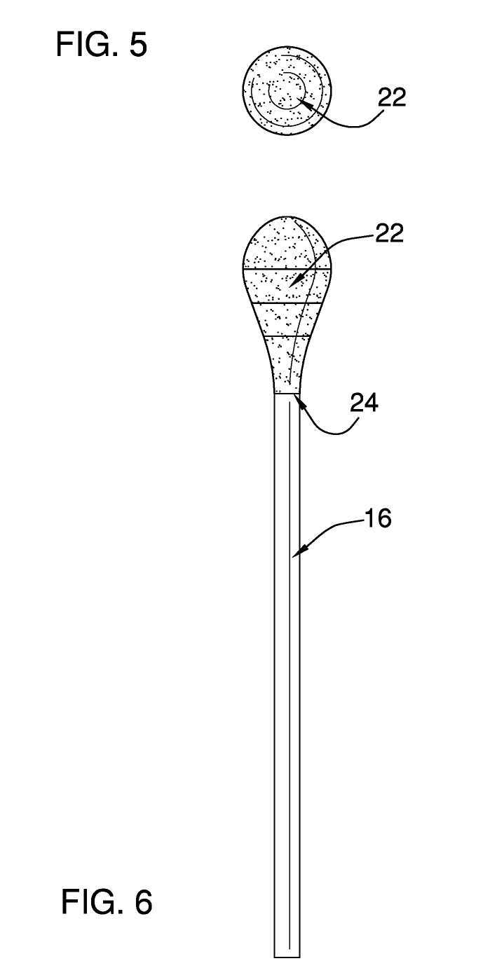

With reference now to the drawings, and in particular to FIGS. 1 through 6 thereof, a new cleaning assembly embodying the principles and concepts of an embodiment of the disclosure and generally designated by the reference numeral 10 will be described.

As best illustrated in FIGS. 1 through 6, the nose cleaning assembly 10 generally comprises at least one rod 12. The at least one rod 12 is configured to be grasped in digits of a hand of a user to selectively position opposing ends 14 of the at least one rod 12 into a nostril of the user. In one embodiment, the at least one rod 12 is circularly shaped when viewed longitudinally. In another embodiment, the at least one rod 12 comprises cardboard. In yet another embodiment, the at least one rod 12 comprises a first stick 16 and a second stick 18.

At least one swab 20 is coupled to a respective opposing end 14 of the at least one rod 12. The at least one swab 20 is configured to absorb mucous that is positioned in the nostril to remove the mucous from the nostril. In one embodiment, the at least one swab 20 is substantially ellipsoid shaped. In another embodiment, the at least one swab 20 comprises a sponge 22 that is coupled to a terminus 24 of the first stick 16.

At least one plate 26 is coupled to a respective opposing end 14 of the at least one rod 12. The at least one plate 26 is configured to scrape hardened deposits that are positioned in the nostril to remove the hardened deposits from the nostril. The at least one plate 26 has a first endpoint 28 that is distal from the at least on rod 12. In one embodiment, the first endpoint 28 is arcuate. In another embodiment, the at least one plate 26 is tapered adjacent to the first endpoint 28. In yet another embodiment, the at least one plate 26 comprises a pair of scrapers 30. The scrapers 30 are coupled singly to opposing termini 32 of the second stick 18.

A tube 34 is coupled to and extends from a second endpoint 36 of the at least one plate 26. The tube 34 is complementary to the at least one rod 12. The tube 34 is positioned to insert the respective opposing end 14 of the at least one rod 12 to couple the plate 26 to the at least one rod 12.

In use, the sponge 22 that is positioned on the first stick 16 is configured to insert into the nostril to absorb the mucous that is positioned in the nostril to remove the mucous from the nostril. The scrapers 30 are positioned on the second stick 18 so that each scraper 30 is configured to selectively insert into the nostril to scrape the hardened deposits that are positioned in the nostril to remove the hardened deposits from the nostril.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A nose cleaning assembly comprising:
at least one rod configured for grasping in digits of a hand of a user selectively positioning opposing ends of said at least one rod for inserting into a nostril of the user;
at least one swab coupled to a respective said opposing end of said at least one rod;
at least one plate coupled to a respective said opposing end of said at least one rod; and
wherein said at least one swab is positioned on said at least one rod such that said at least one swab is configured for absorbing mucous positioned in the nostril for removing the mucous from the nostril, wherein said at least one plate is positioned on said at least one rod such that said at least one plate is configured for scrapping hardened deposits positioned in the nostril for removing the hardened deposits from the nostril.

2. The assembly of claim 1, further including said at least one rod being circularly shaped when viewed longitudinally.

3. The assembly of claim 1, further including said at least one rod comprising cardboard.

4. The assembly of claim 1, further comprising:
said at least one rod comprising a first stick and a second stick;
said at least one swab comprising a sponge coupled to a terminus of said first stick; and
said at least one plate comprising a pair of scrapers, said scrapers being coupled singly to opposing termini of said second stick.

5. The assembly of claim 1, further including said at least one swab being substantially ellipsoid shaped.

6. The assembly of claim 1, further including said at least one plate having a first endpoint distal from said at least on rod, said first endpoint being arcuate.

7. The assembly of claim 6, further including said at least one plate being tapered adjacent to said first endpoint.

8. The assembly of claim 1, further including a tube coupled to and extending from a second endpoint of said at least one plate, said tube being complementary to said at least one rod, wherein said tube is positioned on said at least one rod such that said tube is positioned for inserting said respective said opposing end of said at least one rod for coupling said plate to said at least one rod.

9. A nose cleaning assembly comprising:
at least one rod configured for grasping in digits of a hand of a user selectively positioning opposing ends of said at least one rod for inserting into a nostril of the user, said at least one rod being circularly shaped when viewed longitudinally, said at least one rod comprising cardboard, said at least one rod comprising a first stick and a second stick;
at least one swab coupled to a respective said opposing end of said at least one rod, wherein said at least one swab is positioned on said at least one rod such that said at least one swab is configured for absorbing mucous positioned in the nostril for removing the mucous from the nostril, said at least one swab being substantially ellipsoid shaped, said at least one swab comprising a sponge coupled to a terminus of said first stick;
at least one plate coupled to a respective said opposing end of said at least one rod, wherein said at least one plate is positioned on said at least one rod such that said at least one plate is configured for scrapping hardened deposits positioned in the nostril for removing the hardened deposits from the nostril, said at least one plate having a first endpoint distal from said at least on rod, said first endpoint being arcuate, said at least one plate being tapered adjacent to said first endpoint, said at least one plate comprising a pair of scrapers, said scrapers being coupled singly to opposing termini of said second stick;
a tube coupled to and extending from a second endpoint of said at least one plate, said tube being complementary to said at least one rod, wherein said tube is positioned on said at least one rod such that said tube is positioned for inserting said respective said opposing end of said at least one rod for coupling said plate to said at least one rod; and
wherein said sponge is positioned on said first stick such that said sponge is configured inserting into the nostril for absorbing the mucous positioned in the nostril for removing the mucous from the nostril, wherein said scrapers are positioned on said second stick such that each said scraper is configured for selectively inserting into the nostril for scrapping the hardened deposits positioned in the nostril for removing the hardened deposits from the nostril.
